(19) United States
(12) Patent Application Publication
(10) Pub. No.: US 2005/0228467 A1
(43) Pub. Date: Oct. 13, 2005

(54) Implantable miniature titanium to stainless steel connector

(76) Inventor: Guangqiang Jiang, Santa Clarita, CA (US)

Correspondence Address: ALFRED E. MANN FOUNDATION FOR;SCIENTIFIC RESEARCH, PO BOX 905, 25134 RYE CANYON LOOP, SUITE 200, SANTA CLARITA, CA, 91380, US

(21) Appl. No.: 10/833,588

(22) Filed: Apr. 27, 2004

Related U.S. Application Data
(63) Continuation-in-part of application No. 10/821,023, filed on Apr. 7, 2004.

(51) Int. Cl. A61N 1/05

(52) U.S. Cl. 607/115; 600/373

(57) ABSTRACT

An implantable miniature bonded device comprising a bimetallic electrode hermetically bonded to a ceramic tube that contains electrical components for processing electrical signals that are communicated to or from living tissue via an electrically conductive wire. The ceramic tube is an electrical insulator that is biocompatible and that is bonded to a titanium electrode part, which in turn is bonded to a stainless steel electrode part by brazing. Degradation of the electrical conductivity between the wire and the device is avoided by attaching an electrically conductive wire directly to the stainless steel part, rather than to the titanium part.

### CROSS REFERENCE TO RELATED APPLICATION

This application is a Continuation In Part of co-pending U.S. patent application Brazing Titanium to Stainless Steel Using Laminated Ti—Ni Filler Material, attorney docket number A369-USA, application Ser. No. 10/821,023, filed on Apr. 7, 2004 and incorporated in its entirety herein by reference.

This application is related to but in no way dependent on commonly assigned U.S. patent applications: Manufacturing Method for a Ceramic to Metal Seal, application Ser. No. 10/714,913; Layered Sphere Braze Material, application Ser. No. 10/793,457; Particulate Braze Material, application Ser. No. 10/793,006; Brazing Titanium to Stainless Steel Using Nickel Filler Material, application Ser. No. 10/793,536; and Brazing Titanium to Stainless Steel Using Layered Particulate, application Ser. No. 10/823,963, all incorporated in their entirety herein by reference.

### BRIEF DESCRIPTION OF THE DRAWINGS

FIG. 1 illustrates a side view of the component assembly with filler material as a foil between the stainless steel part and the titanium part.

FIG. 2 schematically depicts the bonding steps of the present invention.

FIG. 3 presents an isometric view of a titanium-nickel laminated filler material having three foil layers.

FIG. 4 presents an isometric view of a titanium-nickel laminated filler material having five foil layers.

FIG. 5 illustrates the compact filler material comprised of discrete particles of titanium and nickel.

FIG. 6 presents a cross-sectional view of a discrete particle of nickel and titanium layers.

FIG. 7 presents an exploded isometric view of a ceramic tube, titanium part, and stainless part.

FIG. 8 illustrates a bonded device with a crimp attached wire.

### DETAILED DESCRIPTION OF THE PREFERRED EMBODIMENT

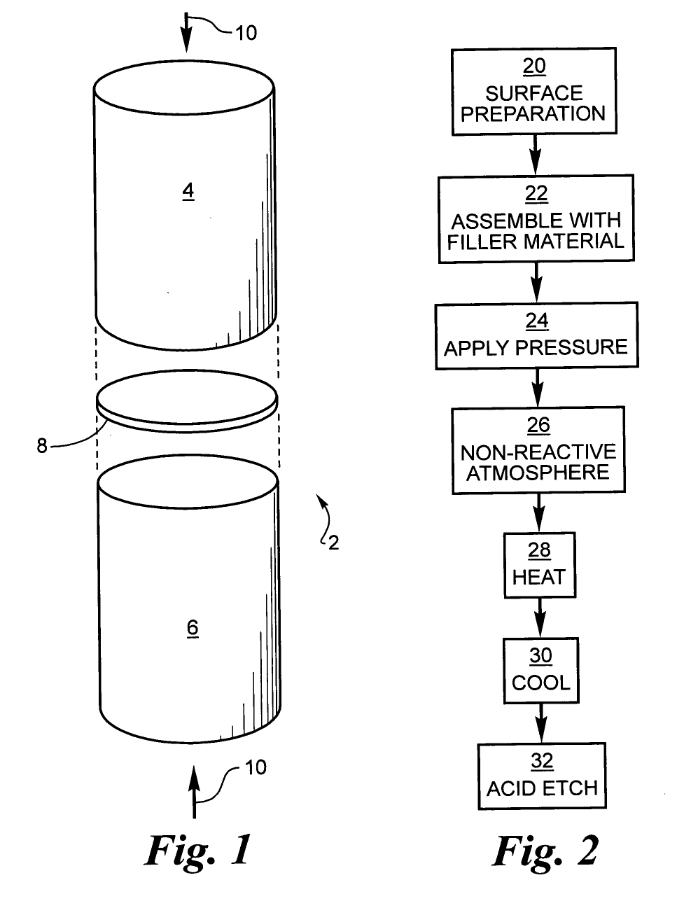

FIG. 1 presents component assembly 2 having a titanium part 4, a stainless steel part 6, and a filler material 8. While the titanium part 4 may be securely attached to stainless steel part 6 by any of several techniques that are known to those skilled in the art, brazing is the preferred method of attachment. In an alternative embodiment, the stainless steel part 6 is attached to titanium part 4 by a threaded connection (not illustrated). Component assembly 2 is heated to a specific process temperature that is below the melting point of titanium part 4 or of the melting point of stainless steel part 6, for a specific period of time, at a pressure that is created by force 10, that is exerted to place filler material 8 in intimate contact with the titanium part 4 and stainless steel part 6.

Filler material 8 is preferably a laminate metal foil having a thickness of approximately ten-thousandths (0.010) of an inch and more preferably less than 0.001 inches. In alternate embodiments, filler material 8 comprises substantially pure nickel, nickel alloys, or titanium-nickel alloys, where the substantially pure nickel is preferably greater than 98% nickel. Filler material 8 is selected from the group of materials that are compatible with the stainless steel chosen for stainless steel part 6 in that they wet the surface during the bonding process and enter into a diffusion process with the stainless steel part 6, thereby creating a strong bond joint during processing. Filler material 8 is further selected from the group of materials that are compatible with the titanium part 4. Filler material 8 forms a bond between titanium part 4 and stainless steel part 6 at the bonding temperature and pressure utilized during processing. The group of filler materials that are compatible with both the stainless steel part 6 and the titanium part 4 includes substantially pure titanium and nickel laminate compositions, preferably comprising filler materials of about 22% to 98% nickel and the balance titanium. In a preferred embodiment, FIG. 3, filler material 8 is preferably comprised of alternating foil layers 12 and 14. Preferably, for example, as shown in FIG. 3, a laminate stack of commercially pure nickel layer 12 on the top outer surface 42 and a similar nickel layer 12 on the bottom outer surface 44. Sandwiched between the nickel layers 12 is a titanium layer 14. The nickel layer 12 having at least 99.0% nickel and less than 1.0% of other elements with a thickness of greater than approximately 0.0003 inches and the titanium layer 14 comprised of commercially pure titanium foil having at least 99.0% titanium and less than 1.0% of other elements with a thickness of greater than approximately 0.0003 inches.

The inventors prefer the term “laminated” versus other descriptive, but equally applicable, terms such as “layered”, “clad”, or “composite” material. The laminated filler material is not an “alloy” of nickel and titanium. An alloy, which is defined as a homogeneous mixture of two or more metals, where the atoms of one replace or occupy interstitial positions between the atoms of the other, of nickel and titanium, for example, does not demonstrate the depressed melting point that is available at a eutectic composition when nickel and titanium are in intimate contact. The laminate material supplies substantially pure nickel to initiate bonding with other metals, such as titanium or stainless steel, for example, at relatively low eutectic temperatures. For example, the lowest liquidus temperature (also referred to herein as the melting point) in the nickel-titanium phase diagram occurs at 28% by weight nickel and is 942° C. Therefore, the optimum braze temperature will be greater than this temperature.

In a further preferred embodiment, FIG. 4, the metal foil layers 15, 15′, and 15″, which are comprised of nickel, are placed in laminated filler material 8 as the top outer surface 42 and as the bottom outer surface 44, thereby making the nickel available to react directly with the stainless steel part 6 and the titanium part 4. Alternating layers of inner mating foil layer 17 and 17′, which are comprised of titanium, are placed between the metal foil layers 15, 15′, and 15″.

It is known to those skilled in the art that the total composition of a laminate stack of alternating nickel and titanium foil is controlled by the thickness of the foil layers, where the volume fraction of nickel and titanium is converted to weight percent by accounting for the density of the nickel and titanium. For example, to achieve a total laminate stack composition of a filler material 8 having a composition of 50 weight percent Ni and 50 weight percent Ti, where the density of nickel is 8.90 g/cc and of titanium is 4.51 g/cc, the thickness of the filler material 8 will be 33.6% Ni foil and 66.4% Ti foil.

Titanium part 4 may be comprised of a titanium alloy and is comprised of Ti-6AI-4V, i.e. an alloy of titanium with 6 weight percent aluminum and 4 weight percent vanadium, in a preferred embodiment. Stainless steel part 6 may be comprised of one of the corrosion resistant stainless steels, such as a 200, 300, or 400 series stainless steel, and in a preferred embodiment stainless steel part 6 comprises 316L stainless steel. This configuration of components offers the advantage of being biocompatible and of being capable of forming hermetic seals.

In an alternate embodiment, rather than using filler material 8 as a foil, filler material 8 may be a thin coating that is applied to the bonding surface of either the titanium part 4 or stainless steel part 6 by any of a variety of chemical processes, such as electroless plating and electroplating, or by any of a variety of thermal processes, such as sputtering, evaporating, or ion beam enhanced deposition.

In another embodiment, filler material 8 is applied as a thin coating of metallic beads, metallic powder, or discrete particles. The coating may be applied in any of several methods known to those skilled in the art, such as painting, spraying, or dipping. The applied coating consists of discrete particles of nickel and of titanium that aid in bonding the stainless steel part 6 and the titanium part 4 during the braze process.

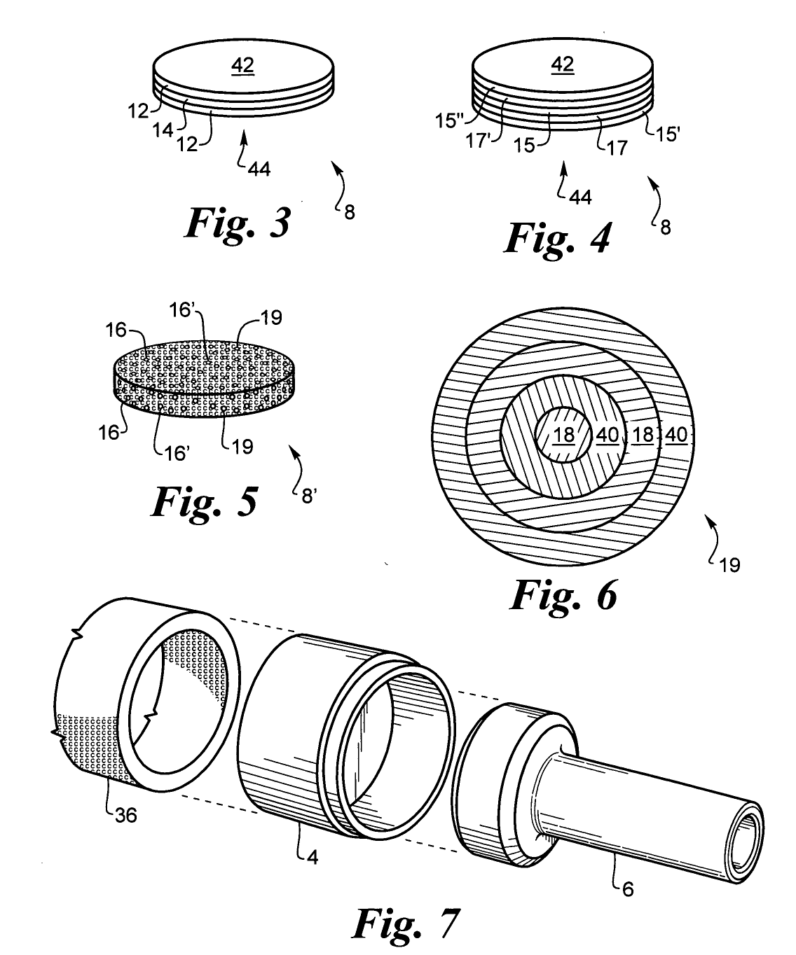

In a further alternate embodiment, a compact filler material 8′, FIG. 5, is comprised of a bonded compact of primary alloy particulate 16 and secondary alloy particulate 16′, where primary alloy particulate 16 is preferably comprised of a nickel alloy and primary alloy particulate 16′ preferably comprises a titanium alloy. The compact filler material 8′ is formed by any of several techniques that are known to one skilled in the art, including cold pressing, warm pressing, slurry preparation, etc. The intimate mixture of primary alloy particulate 16 and secondary alloy particulate 16′ bond together as well as react with the stainless steel part 6 and the titanium part 4 during the braze operation to yield a bonded component assembly 2.

Another alternate embodiment of forming a bonded component assembly 2 utilizes the compact filler material 8′, as presented in FIG. 5, that is comprised of layered discrete particle 19, preferably spheres, comprises a layered or laminated composition, as shown in FIG. 6. In a preferred embodiment, layered discrete particle 19 comprises alternating layers of primary particle laminate layer 18 and secondary particle laminate layer 40, where primary particle laminate layer 18 preferably comprises a nickel alloy, preferably substantially pure nickel, and secondary particle laminate layer 40 comprises a titanium alloy, preferably substantially pure titanium. The overall bonding methods and processes are analogous to those employed for the several embodiments. The compact filler material 8′ is formed either by conventional densification processes, such as cold pressing the particulate into a preform, FIG. 5, or the compact filler material 8′ is formed in situ between the parts being bonded by applying the particulate to the metal parts being bonded prior to assembling them for brazing.

The process steps that are employed to create component assembly 2 with a strong bond between titanium part 4 and stainless steel part 6 are schematically represented in FIG. 2. First, the surfaces to be bonded are prepared in step 20 by machining to assure that they will intimately conform to each other during bonding. The surfaces are smoothed and cleaned.

In step 22, component assembly 2 is prepared with filler material 8 between titanium part 4 and stainless steel part 6. In step 24, force 10 is applied to compress filler material 8 between titanium part 4 and stainless steel part 6. Force 10 is sufficient to create intimate contact between the parts. Force 10 is applied to assure that a bond is formed between titanium part 4 and stainless steel part 6, thus creating a hermetic seal between the two parts. It is preferred that the resulting pressure be greater than about five psi.

In step 26, the assembly to be heat processed is placed in a furnace in a non-reactive atmosphere, which is preferably vacuum, but which, in an alternative embodiment, can be any of several atmospheres that are known to those skilled in the art, such as argon, nitrogen or hydrogen. A non-reactive atmosphere is applied before the furnace is heated to the processing temperature in step 28. A preliminary holding temperature may be utilized to allow the thermal mass of the parts to achieve equilibrium before proceeding with heating. In a preferred embodiment, the vacuum is less than 10⁻⁵ torr, to assure that the filler material 8 and titanium part 4 do not oxidize. Component assembly 2 is held at the selected temperature, which is between approximately 940° and 1260° C., for approximately 5 to 60 minutes, while force 10 continues to be exerted on filler material 8. The exact time, temperature and pressure are variable with each other so as to achieve a strong bond between titanium part 4 and stainless steel part 6. For example, in a preferred embodiment, a 316L stainless steel part is bonded to a Ti-6AI-4V part in vacuum at 10⁻⁶ torr at approximately 1000° C. for 10 minutes with a pressure of about 50 psi on a nickel-titanium foil of approximately 0.002 inches total thickness.

The furnace is cooled and component assembly 2 is cooled to room temperature in step 30. In optional step 32, component assembly 2 is cleaned by being placed in a bath, after thermal processing is complete, to assure removal of all nickel and nickel salts. This bath is preferably an acid bath that etches the exposed surfaces of component assembly 2. In a preferred embodiment, the bath is nitric acid. Removal of nickel and nickel salts in the etch bath insures that component assembly 2 is biocompatible. Nickel and nickel salts are detrimental to living animal tissue. It is preferred that all of the nickel that is introduced as filler material 8 is combined with the titanium and is chemically tied up by thermal processing to be unavailable as free nickel or as a nickel salt. Component assembly 2 is biocompatible after bonding and processing.

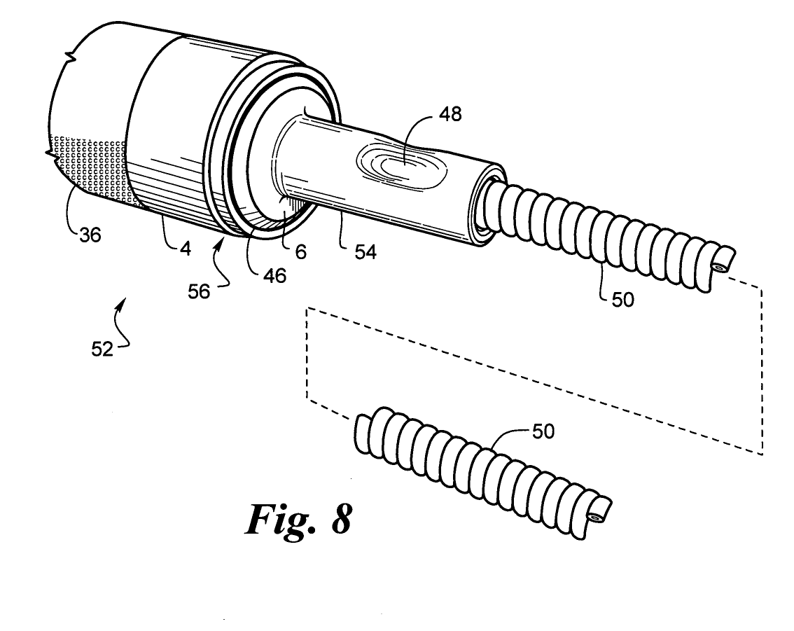

In a preferred embodiment, component assembly 2 is either an electrical sensor or an electrical stimulator that is implanted in a human body, although it could equally well be implanted in any animal. It must survive long periods in the hostile environment of a living body, which is basically a warm saline solution. In a preferred embodiment, component assembly 2 is either a sensor or stimulator comprised of a hollow ceramic tube 36, containing various electronic components, that is bonded to a titanium part 4. The titanium part 4 and the stainless steel part 6 comprising an electrode 56 for communicating electrical signals between living tissue and the bonded device 52. The component assembly must be watertight, resisting salt-water intrusion as well as growth of living tissue into the titanium-to-stainless steel braze joint. FIG. 7 presents an exploded isometric view of a ceramic tube 36 that is bonded to a titanium part 4 and a stainless steel part 6. The stainless steel part 6 is designed to accept an electrically conductive wire 50, for transmission of electrical signals.

Further, component assembly 2 does not corrode while implanted in the body. The materials are chosen such that post-bonding they are not susceptible to corrosion either individually or in the as-bonded state. Component assembly 2 resists electrolytic corrosion as well as crevice corrosion, because of the materials selected for construction of component assembly 2.

A bonded device 52 is presented in FIG. 8 wherein a ceramic tube 36 is bonded to titanium part 4 which is bonded to stainless steel part 6 with a filler material 8 at braze joint 46. Stainless steel part 6 contains a receiver 54 into which a wire 50 is inserted and attached, preferably by crimping, such that crimp indentation 48 retains wire 50. The bonded device 52 provides good electrical conductivity via stainless steel part 6 connecting to wire 50. Stainless steel part 6 is brazed to titanium part 4 that is bonded by known methods to ceramic tube 36.

The bonded device 52 is preferably an implantable stimulator of muscle or nerve when implanted in living tissue, although it may operate as a sensor also. In a preferred embodiment, the device 52 has an outside diameter of 6 mm or less, enabling such a miniature device, which preferably has an overall length of less than about 60 mm, to be inserted in living tissue by injection, rather than by conventional surgical techniques. Ceramic tube 36 is an electrically insulating case, which typically is hollow and contains an electronics package and a power source, such as a battery, capacitor, magnetic field to electricity converter, and electrically conductive case ends described here as titanium part 4 and stainless steel part 6, forming an electrically conductive electrode 56 which conducts electrical signals from the stimulator and/or to a sensor, depending upon the design and function of that particular miniature bonded device 52. Bonded device 52 has at least one electrode, but preferably it has 2 to 8 or more electrodes, depending upon its particular design and function, although, for illustrative purposes, only one electrode is presented in FIG. 8.

Insulating case 4 contains the electronics, which may include a battery or other energy storage device and signal generating or receiving circuitry and is made of an electrically insulating ceramic material that is capable of being hermetically sealed and that is also biocompatible. The ceramic may be alumina, glass, titania, zirconia, stabilized-zirconia, partially-stabilized zirconia, tetragonal zirconia, magnesia-stabilized zirconia, ceria-stabilized zirconia, yttria-stabilized zirconia, or calcia-stabilized zirconia, and in a preferred embodiment, ceramic tube 36 is yttria-stabilized zirconia, although other ceramic materials may also be used. The ceramic tube 36 is preferably biocompatible as well as capable of being hermetically sealed, to prevent permeation of bodily fluids into the case.

The bond joints between the ceramic tube 36 and titanium part 4 and stainless steel 6 are preferably biocompatible, non-corrosive, and hermetic. To this end, it has been discovered that attaching an electrically insulating wire 50, which is suitable to conduct a signal to or from a remote location, to bonded device 52 presents a challenge when the wire was attached directly to titanium part 4 by, for example, by crimping or any of the methods known to one skilled in the art. The titanium part 4 develops an electrically insulating oxide layer that lowers the efficiency of the device and decreases useful life if an on-board battery is employed to power the device.

The solution is to employ stainless steel part 6 as the attachment electrode for wire 50. As presented in FIG. 8, in a preferred embodiment wire 50 is inserted into receiver 54, which is part of stainless steel part 6. The stainless steel does not corrode and provides a long-lived, low-resistance electrical contact with wire 50. The illustrated attachment method employs forming a crimp indentation 48 on receiver 54 that, in turn, secures electrically conductive wire 50 to stainless steel part 6.

In a preferred embodiment, wire 50 is a highly conductive metal that is also benign in the body, such as MP35 alloy, although stainless steel, such as 316L, platinum, or an alloy of platinum-iridium may be employed. Preferably, the wire has a diameter of approximately 0.030 inches. It is contained in a wire insulator to electrically isolate it from the body tissue and fluids and, in a preferred embodiment, the wire insulator is comprised of TEFLON, which is optionally coated with silicone.

Forming a strong, biocompatible bond between stainless steel part 6 and titanium part 4 was accomplished by forming braze joint 46 by utilizing a nickel-titanium or substantially pure nickel braze material.

These various embodiments are of devices and methods for connecting an electrically conductive wire 50 to a miniature implantable stimulator via a stainless steel to titanium electrode connector, in order to efficiently transmit or receive an electrical signal that is associated with the implantable stimulator.

Obviously, many modifications and variations of the present invention are possible in light of the above teachings. It is therefore to be understood that, within the scope of the appended claims, the invention may be practiced otherwise than as specifically described.

1. An implantable device suitable for use in living tissue and adapted for sensing or stimulation in such living tissue, comprising:
a hollow ceramic tube;
at least one titanium part hermetically bonded to said ceramic tube and forming a part of an electrically conductive electrode for communicating electrical signals between the device and living tissue; and
a stainless steel part secured to said at least one titanium part, thereby completing said electrode, and further comprising a receiver that accepts an electrically conductive wire.

2. The component assembly of claim 1, wherein said hollow ceramic tube comprises yttria-stabilized zirconia.

3. The component assembly of claim 1, wherein said titanium part comprises Ti-6AI-4V.

4. The component assembly of claim 1, wherein said stainless steel part comprises 316L stainless steel.

5. The component assembly of claim 1, wherein said implantable device comprises a cylinder less than about 60 mm in length and less than about 6 mm in diameter.

6. The component assembly of claim 1, wherein said stainless steel part is brazed to said at least one titanium part.

7. The component assembly of claim 1, wherein said stainless steel part is brazed to said at least one titanium part by use of a nickel-titanium filler material.

8. The component assembly of claim 1, wherein said stainless steel part is brazed to said at least one titanium part by use of a substantially pure nickel filler material.

9. The component assembly of claim 1, wherein said stainless steel part comprises a corrosion resistant stainless steel.

10. The component assembly of claim 1, wherein said electrically conductive wire comprises an MP35 alloy.

11. The component assembly of claim 1, wherein said electrically conductive wire comprises a 316L stainless steel.

12. The component assembly of claim 1, wherein said electrically conductive wire comprises platinum.

13. The component assembly of claim 1, wherein said electrically conductive wire comprises a platinum-iridium alloy.
